(19) United States
(12) Patent Application Publication
(10) Pub. No.: US 2024/0082478 A1
(43) Pub. Date: Mar. 14, 2024

(54) EAR IRRIGATION DEVICE

(71) Applicant: Edward Wagner, Portland, OR (US)

(72) Inventor: Edward Wagner, Portland, OR (US)

(21) Appl. No.: 17/969,665

(22) Filed: Oct. 19, 2022

Related U.S. Application Data
(60) Provisional application No. 63/404,804, filed on Sep. 8, 2022.

(51) Int. Cl. A61M 3/02

(52) U.S. Cl. CPC A61M 3/0283; A61M 2210/0662

(57) ABSTRACT

An ear irrigation device for flushing an ear canal to remove debris safely and without the risk of over-insertion. The ear irrigation device includes a disk that has a top and a bottom. A conical protrusion extends from a central top portion of the disc. A tubular member extends from a central bottom portion of the disc. The tubular member has a lure lock to connect with the irrigation fluid delivery system. A bore extends through the conical protrusion, the disc, and opens into the tubular member, wherein the bore forms a channel through which a stream of water can pass through and eject from the tip of the conical protrusion. The conical protrusion can insert into an orifice while the disc prevents over-insertion. Multiple drain holes provided in the conical protrusion can channel out the irrigation fluid flushed from the ear canal.

### CROSS-REFERENCE TO RELATED APPLICATIONS

This application claims priority from a U.S. Provisional Patent Appl. No. 63/404,804 filed on Sep. 8, 2022, which is incorporated herein by reference in its entirety.

### FIELD OF INVENTION

The present invention relates to a device for ear irrigation, and more particularly, the present invention relates to a device for ear irrigation that is safe to use.

### BACKGROUND

Ear irrigation tips are known in the art for flushing ear debris from the ear canal. The ear debris can be deposited cerumen, infectious secretions, and the like. Known ear irrigation tips are tubular members that can be connected to a water delivery system for directing a stream of water into the ear canal. However, such ear irrigation tips have some major drawbacks, such as a stream of water can result in pressure build-up that may harm the sensitive and fragile tympanic membrane or can cause pain in the ear canal. The tip of the elongated tube can also harm the sensitive tissue in the ear canal.

Thus, a need for an ear irrigation device is appreciated for safer flushing of the ear canal.

### SUMMARY OF THE INVENTION

The following presents a simplified summary of one or more embodiments of the present invention to provide a basic understanding of such embodiments. This summary is not an extensive overview of all contemplated embodiments and is intended to neither identify critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to an ear irrigation device that is safe to use.

It is another object of the present invention that the device allows the removal of irrigation fluid from the ear canal.

It is still another object of the present invention that the device does not over insert into the ear canal.

It is yet another object of the present invention that the device is made of hypoallergic materials.

It is a further object of the present invention that the device does not cause laceration of the ear canal.

It is still a further object of the present invention that the ear debris can be effectively removed.

In one aspect, disclosed is an ear irrigation device comprising a disc that has a top and a bottom; a conical protrusion extends perpendicular from a central top portion of the disc, an end of the conical protrusion defines a proximal end of the ear irrigation device; a tubular member extends from a central bottom portion of the disc, wherein the tubular member and the conical protrusion are coaxial, wherein the tubular member is hollow, an end of the tubular member defines a distal end of the ear irrigation device; an interlocking mechanism configured in the tubular member, wherein the interlocking mechanism is configured for coupling the ear irrigation device to a water delivery system; a bore extends between the proximal end and the distal end of the ear irrigation device, wherein the bore extends through the conical protrusion, the disc, and opens into the tubular member, the bore forms a channel through which a stream of water can pass and eject from the proximal end of the ear irrigation device; and a plurality of drain holes that ends from a mid of the conical protrusion and through the disc away from the tubular member.

In one implementation, the ear irrigation device is made of hypoallergic material. The hypoallergic material is rubber. The disc is of a round and planar profile, a diameter of the disc is about 1.5 inches, a largest diameter of the conical protrusion is about 0.5 inches. The interlocking mechanism is a lure lock. The plurality of drain holes comprises four drain holes configured around the bore. The ear irrigation device is configured to eject a single stream of irrigation fluid through the bore.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a perspective view of an ear irrigation device, according to an exemplary embodiment of the present invention.

FIG. 2 is a side view of the ear irrigation device, according to an exemplary embodiment of the present invention.

### DETAILED DESCRIPTION

Subject matter will now be described more fully hereinafter. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as apparatus and methods of use thereof. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

The following detailed description is described with reference to the drawings, wherein like reference numerals are used to refer to like elements throughout. In the following description, for purposes of explanation, specific details may be outlined in order to provide a thorough understanding of the subject innovation. It may be evident, however, that the claimed subject matter may be practiced without these specific details. In other instances, well-known structures and apparatus are shown in block diagram form in order to facilitate describing the subject innovation. Moreover, the drawings may not be to scale.

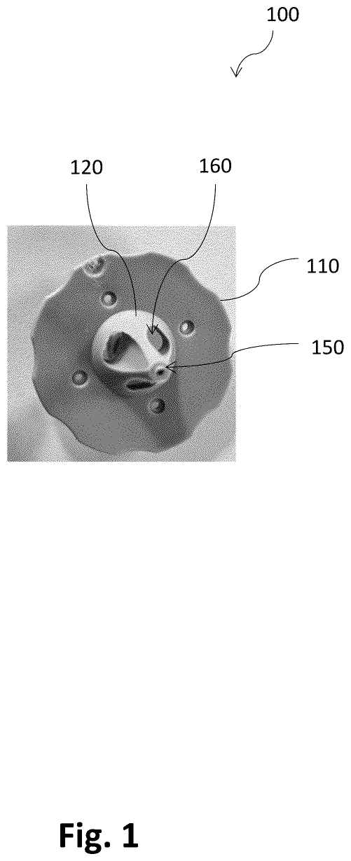

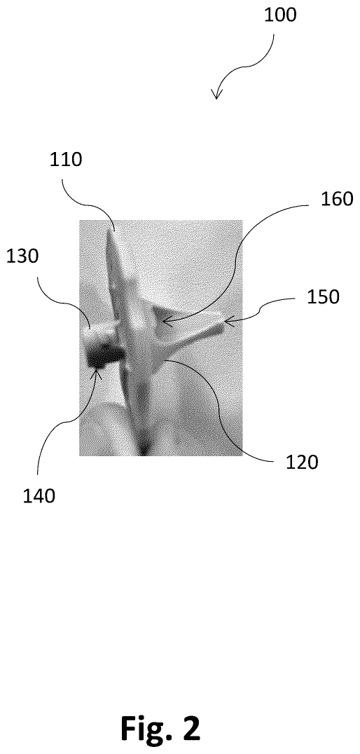

Disclosed is an ear irrigation device that can be used to flush the ear canal with a stream of irrigation fluid without causing pressure build-up or damage to sensitive tissues. The ear irrigation device can be safe to use and can effectively flush debris out from the ear canal. The disclosed ear irrigation device includes a disc that limits the insertion of the ear irrigation device into an orifice. FIGS. 1 and 2 show an exemplary embodiment of the ear irrigation device 100 which has a planar round disc 110 of a diameter at least larger than an opening of the ear canal. The disc prevents over-insertion of the ear irrigation device to prevent damage to any sensitive tissue. The disc is shown as a round shape in FIGS. 1 and 2 for illustration only, and the disc can be made in a round or elliptical shape, and any shape of the disc is within the scope of the present invention.

The ear irrigation device further includes a conical protrusion 120 that perpendicularly extends from a top central portion of the disc 110. The conical protrusion has a wider base integral with the disc while the narrow end of the conical protrusion forms a proximal end of the ear irrigation device. The proximal end of the ear irrigation device is smooth to prevent any damage to the tissue of the ear canal. The conical protrusion can be inserted into the orifice of the ear canal while the disc prevents over-insertion. Thus, the disc is critical for the ensuring safety of the irrigation process.

A tubular member 130 extends perpendicularly from the bottom central portion of the disc. The tubular member can be a hollow tube configured with a lure lock 140 that can be sealably coupled to a water delivery system. The water delivery system, such as an electronic or manual water pump that can deliver water under pressure can be coupled to the ear irrigation device for irrigating the ear canal. It is understood that water or any irrigation fluid can be used. Lure locks are standard interlocking interfaces in medical devices, however, any other interlocking means for coupling the disclosed ear irrigation device to a water delivery system is within the scope of the present invention. The free end of the tubular member forms a distal end of the ear irrigation device.

A bore 150 extends between the proximal end and the distal end of the ear irrigation device. The bore axially extends through the conical protrusion and the disc. The bore form a channel through which a stream of water passes from the water delivery system into the ear canal for irrigation. One end of the bore opens at the tip of the conical protrusion i.e., the proximal end from where the steam of water under pressure can eject. The other end of the bore opens into the hollow member through which water can enter. A single stream of irrigation fluid can eject from the ear irrigation device into the ear canal.

The disclosed ear irrigation device can further include multiple drainage channels 160 that extend from the mid of the conical protrusion and through the disc and opens at the bottom side of the disc projecting away from the tubular member. The irrigation fluid after flushing through the ear canal can drain out through these drain channels. Four drain channels which appear like elongated apertures are shown in FIG. 1. The bore extends axially through the center of the ear irrigation device while the drainage channels can be made around the bore. The shape and dimension of the drainage channels allow the irrigation fluid to exit from the ear canal preventing any pressure build-up. The debris can flush with the irrigation fluid and flow out through the drainage channels. The number of such drainage channels can be varied without departing from the scope of the present invention.

The disclosed ear irrigation device can be made from any hypo-allergic material, such as rubber, silicon, plastic, and the like. In one implementation, the outer diameter of the disc can be about 1-2 inches, and more preferably can be about 1.5 inches. The largest diameter of the conical protrusion can be about 1-2 inches, and more preferably about 0.5 inches. It is understood that the dimensions of the disclosed ear irrigation device can be varied without departing from the scope of the present invention. Perhaps the dimensions of the disclosed ear irrigation device may vary based on several factors such as the age of the patient.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. An ear irrigation device comprising:
a disc that has a top and a bottom;
a conical protrusion extends perpendicular from a central top portion of the disc, an end of the conical protrusion defines a proximal end of the ear irrigation device;
a tubular member extends from a central bottom portion of the disc, wherein the tubular member and the conical protrusion are coaxial, wherein the tubular member is hollow, an end of the tubular member defines a distal end of the ear irrigation device;
an interlocking mechanism configured in the tubular member, wherein the interlocking mechanism is configured for coupling the ear irrigation device to a water delivery system;
a bore extends between the proximal end and the distal end of the ear irrigation device, wherein the bore extends through the conical protrusion, the disc, and opens into the tubular member, the bore forms a channel through which a stream of water can pass and eject from the proximal end of the ear irrigation device; and
a plurality of drain holes that ends from a mid of the conical protrusion and through the disc away from the tubular member.

2. The ear irrigation device according to claim 1, wherein the ear irrigation device is made of hypoallergic material.

3. The ear irrigation device according to claim 2, wherein the hypoallergic material is rubber.

4. The ear irrigation device according to claim 1, wherein the disc is of a round and planar profile, a diameter of the disc is about 1.5 inches, a largest diameter of the conical protrusion is about 0.5 inches.

5. The ear irrigation device according to claim 1, wherein the interlocking mechanism is a lure lock.

6. The ear irrigation device according to claim 1, wherein the plurality of drain holes comprises four drain holes configured around the bore.

7. The ear irrigation device according to claim 1, wherein the ear irrigation device is configured to eject a single stream of irrigation fluid through the bore.
